(19) United States
(12) Patent Application Publication
(10) Pub. No.: US 2018/0325814 A1
(43) Pub. Date: Nov. 15, 2018

(54) CONFECTIONERY FOR ORAL CARE APPLICATIONS

(71) Applicant: The Hershey Company, Hershey, PA (US)

(72) Inventors: Steven M. Kumiega, Hummelstown, PA (US); Paula M. Gibson, Harrisburg, PA (US); Theresa R. Cea, Brooklyn, NY (US); Mary Lou Cunningham, Wilmington, DE (US); Joan L. Apgar, Hummelstown, PA (US); Ian Fairs, Palmyra, PA (US); Balaji Santhanam, Harrisburg, PA (US); Brian J. Paul, Millersburg, PA (US); Jordana Langiotti Swank, Palmyra, PA (US)

(21) Appl. No.: 15/980,296

(22) Filed: May 15, 2018

Related U.S. Application Data
(60) Provisional application No. 62/506,257, filed on May 15, 2017.

(51) Int. Cl. A61K 9/00; A61K 9/20; A61K 47/26; A61K 31/198; A61K 31/661; A61Q 11/00; A23G 3/36; A23G 3/54

(52) U.S. Cl. CPC A61K 9/0056; A23V 2250/6422; A61K 47/26; A61K 31/198; A61K 31/661; A61Q 11/00; A23G 3/36; A23G 3/54; A23G 2200/06; A23V 2002/00; A23V 2200/312; A23V 2250/0606; A23V 2250/6402; A23V 2250/6406; A23V 2250/6418; A23V 2250/642; A61K 9/2095

(57) ABSTRACT

The present invention is directed to a confectionery capable of imparting oral care benefits to an end-user, the confectionery containing: a first sweetener; an optional second sweetener, a flavorant and optionally a functional active ingredient for promoting tooth mineralization, wherein the confectionery is substantially sugar-free. The present invention is also directed to a method of imparting oral care benefits to a consumer involving inserting the above-described confectionery into a consumer's mouth and allowing the confectionery to dissolve in the consumer's mouth over a prolonged period of time.

### FIELD OF THE INVENTION

The present invention relates to confectionery products that are long-lasting and provide oral care benefits.

### BACKGROUND

It is well known that foods containing sugar are to be consumed in moderation. In certain cases, sugary foods may have contributing effects regarding ailments, such as diabetes, hypertension, coronary heart disease, arterial sclerosis and dental cavities. Increasingly, consumers prefer “sugar-free” products as a healthier alternative to gums and candies that contain traditional sugars, such as sucrose, fructose or glucose.

Presently, varieties of confectionery products on the market comprise low calorie alternatives to sugar, but are not targeted to remineralization of tooth enamel. For example, some sugar-free confectionery products, such as sugar-free hard candies that are made of sugar substitutes, lack long-lasting flavor and/or oral care benefits. As used herein, “confection” and “confectionery” means consumable lozenges, mints, and other consumable oral products that are designed or intended to be fully consumed by a user, whether by dissolving or chewing or combinations thereof, as contrasted with “gum” or “gums” which include a portion that does not dissolve and is either spat out or ingested as an undissolved amorphous solid from a consumer's oral cavity. For clarity, as used herein, “confection” and “confectionery” excludes chewing gums and bubble gums, for example.

Prior art breath mints provide sweetness using non-cariogenic sugar alcohols, such as xylitol, sorbitol, or Isomalt. While commercial confectionery products often have ingredients that freshen breath and address dry mouth, these products do not yield significant oral care benefits, such as preventing demineralization of tooth enamel or promoting remineralization of carious lesions.

Edibles, chewables and confections have the potential of being an effective vehicle for delivering beneficial agents to teeth because they permit contact of the agent to the teeth with minimal effort on the part of a patient. Confections such as mints have the advantage of requiring less movement or effort from the consumer because they do not require chewing. Chewing gums which promote remineralization of demineralized teeth are known in the art (such as in U.S. Pat. No. 5,378,131), but not confections. It is surmised that prior art confections suffer from having a shorter residence time in the consumer's mouth than chewing gum. Despite the desirability of confections as a vehicle for delivering beneficial agents to teeth, no effective embodiments of long-lasting confections which promote remineralization have been developed in the art.

Saliva is known to promote good oral care due to its ability to wash out the mouth and thus help remove any cavity-causing food particles that accumulate after eating. Saliva's mineral ions can even promote repair of small lesions in tooth enamel, commonly referred to as remineralization of carious lesions. Confections containing ingredients with the ability to increase salivary flow can aid in the remineralization process. However, the confections of the prior art lack the ability to effectively stimulate salivary flow for time periods comparable to chewing gum, again attributable to their shorter residence time in mouth. Despite the desirability of confections which can stimulate salivary flow, no effective embodiments of long-lasting confections capable of stimulating salivary flow have been developed in the art.

Calcium and phosphate ions have been shown to prevent the dissolution of tooth enamel and to promote the remineralization of carious lesions. Prior art confectionery compositions have been developed comprising multiple ingredients to contribute calcium ions and phosphate ions for oral care. It is suggested in the art that, to be effective for remineralization, calcium and phosphate ions must be provided in combination. Known confectionary compositions required multiple ingredients to provide sources of calcium ions and phosphate ions. The multiple ingredients add material cost to the product, may complicate the manufacturing process, and may alarm consumers concerned about the number of chemical additives listed on a product's label.

There is an unmet and continuing need for a sugar-free confectionery that is long-lasting, promotes high production of saliva, and provides oral care benefits, such as remineralization of demineralized teeth, using a minimal number of active ingredients.

### BRIEF SUMMARY OF THE INVENTION

The present invention is directed to a confectionery capable of imparting oral care benefits to an end-user. In some embodiments the confectionery contains: a first area containing a first sweetener; and a second area containing a second sweetener, a flavorant and optionally at least one functional active ingredient, wherein the first sweetener has a faster dissolution rate than the second sweetener, and the confectionery is substantially sugar-free. In other embodiments, a pressed tablet confectionery comprises a sweetener, a flavorant and optionally at least one functional active ingredient.

The present invention is also directed to a method of imparting oral care benefits to a consumer involving inserting the above-described confectionery into a consumer's mouth and allowing the confectionery to dissolve in the consumer's mouth over a period of from about 5 to 10 minutes.

### BRIEF DESCRIPTION OF THE DRAWINGS

The confectionery according to the present invention is further described with reference to the accompanying drawing, in which:

FIG. 1 is a chart showing the results of in situ hardness testing of two formulations evaluated during the development of the present invention.

FIG. 2 is a chart showing the results of in vitro hardness testing of calcium salts evaluated during the development of the present invention.

FIG. 3 is a chart showing the results of in vitro hardness testing of active ingredients evaluated during the development of the present invention.

FIG. 4 is a chart showing the results of salivary flow and time in mouth testing conducted on sugar alcohols during the development of the present invention.

FIG. 5 is a chart of in situ hardness testing results of three formulations on sound tooth enamel and lesioned tooth enamel evaluated during the development of the present invention.

### DETAILED DESCRIPTION OF THE PREFERRED EMBODIMENT

The ensuing detailed description provides preferred exemplary embodiments only, and is not intended to limit the scope, applicability, or configuration of the herein disclosed invention. Rather, the ensuing detailed description of the preferred exemplary embodiments will provide those skilled in the art with an enabling description for implementing the preferred exemplary embodiments in accordance with the herein disclosed invention. It is understood that various changes may be made in the function and arrangement of elements without departing from the spirit and scope of the invention, as set forth in the appended claims.

To aid in describing the invention, directional terms may be used in the specification and claims to describe portions of the present invention (e.g., upper, lower, left, right, etc.). These directional definitions are merely intended to assist in describing and claiming the invention and are not intended to limit the invention in any way. In addition, reference numerals that are introduced in the specification in association with a drawing figure may be repeated in one or more subsequent figures without additional description in the specification, in order to provide context for other features.

Each percentage provided in the specification and claims should be understood to represent a percentage on a weight percentage basis unless specifically indicated otherwise.

The term “substantially sugar-free”, as used herein, means that the confectionery in question contains less than 1 percent by weight of sugar. As used herein, the term “sugar” means aldoses and ketoses which are mono- or disaccharides, including but not limited to erythrose, threose, ribose, arabinose, xylose, lyxose, allose, altrose, glucose, mannose, gulose, idose, galactose, talose, erythrulose, ribulose, xyulose, psicose, fructose, sorbose, tagatose, maltose, lactose, sucrose and the like.

The term “functional active ingredient” as used herein, means a substance added to the confectionery which, when released into an end-user's mouth, will have a beneficial effect on the user. Exemplary functional active ingredients include, but are not limited to: calcium sources, phosphate sources, compounds which modulate bacterial activity, biofilm dispersing agents, biomimetic materials and fluoride.

The term “area” as used herein means a distinct quantity, region or thickness of material, or a portion of a whole. The term “area” as used herein includes any layer, zone, segment or part of material. The area may be a continuous region or may be comprised of more than one distinct region. The area has a composition that may differ from other parts of the whole, and retains this composition. In other words, the area is not blended with other parts of the whole material to form a homogenous mixture.

It is desirable to have a confectionery that has a pleasing flavor, stimulates production of saliva, and enhances remineralization of teeth. It is known in the art that chewing gum provides oral care benefits including remineralization of tooth surfaces (See, e.g. U.S. Pat. No. 5,378,131). Chewing gum's ability to increase salivary flow is thought to be a mechanism by which remineralization occurs. Comparable benefits from a confection have not been observed. Prior art lozenges are not long-lasting and fail to provide remineralization and increased saliva production. The inventors of the present application have discovered that positive oral care benefits are realized by an end-user by providing a confectionary capable of inducing in a user, in vivo, a large and rapid spike in saliva production, and thereafter maintaining a steady, long-lasting flow of saliva production and optionally delivering one or more functional active ingredients to an end-user's mouth.

Extensive developmental experimentation was conducted to evaluate the oral care benefits and processability of various formulations of ingredients. The present inventors discovered confectionary products having the desired beneficial oral care properties which can also be manufactured efficiently and used easily by consumers.

The first step in the development of the products was identifying formulations able to promote salivary flow in order to take advantage of the known oral cleansing and remineralization benefits of saliva. Included in this approach was the hypothesis that since salivary flow is an important factor in tooth remineralization, increasing product “time in mouth” (TIM) and salivary flow would contribute to positive oral care and remineralization.

A benchmarking study was conducted to compare the effect on salivary flow of multiple formulations including gum, pressed tablet confections, extruded confectionery mints, and existing commercial confectionery products. At least thirty formulations containing different ingredients and mixtures of ingredients were evaluated. Based on the results of this study, an inventive extruded confectionery formulation containing xylitol was found to promote salivary flow to a greater extent than chewing gum.

Two formulations containing xylitol were then developed to evaluate different strategies for stimulating salivary flow using different forms of confection. The first formulation was a dual layer confection where a first layer containing xylitol was designed to dissolve rapidly, providing an initial spike in salivary flow, while a second layer containing Isomalt and a cellulosic binder was designed to dissolve more slowly to extend TIM and thereby lengthen the period of increased salivary flow. A second formulation contained xylitol and Isomalt combined in a pressed tablet form. These formulations were evaluated for their ability to prevent demineralization of sound teeth and promote remineralization of demineralized teeth.

An in situ clinical trial was developed using sound and demineralized bovine tooth samples. Hardness measurements of the tooth sample surfaces were taken at the beginning of the study using a microhardness tester to measure the depth of an indentation produced in the tooth sample under a standardized load and for a controlled duration. Each of the formulations was then exposed to a tooth sample under controlled conditions. A control sample which was not exposed to any formulation was also included for comparison. Hardness measurements were taken after the exposure and compared to the initial measurements to assess the effect on mineralization of each formulation.

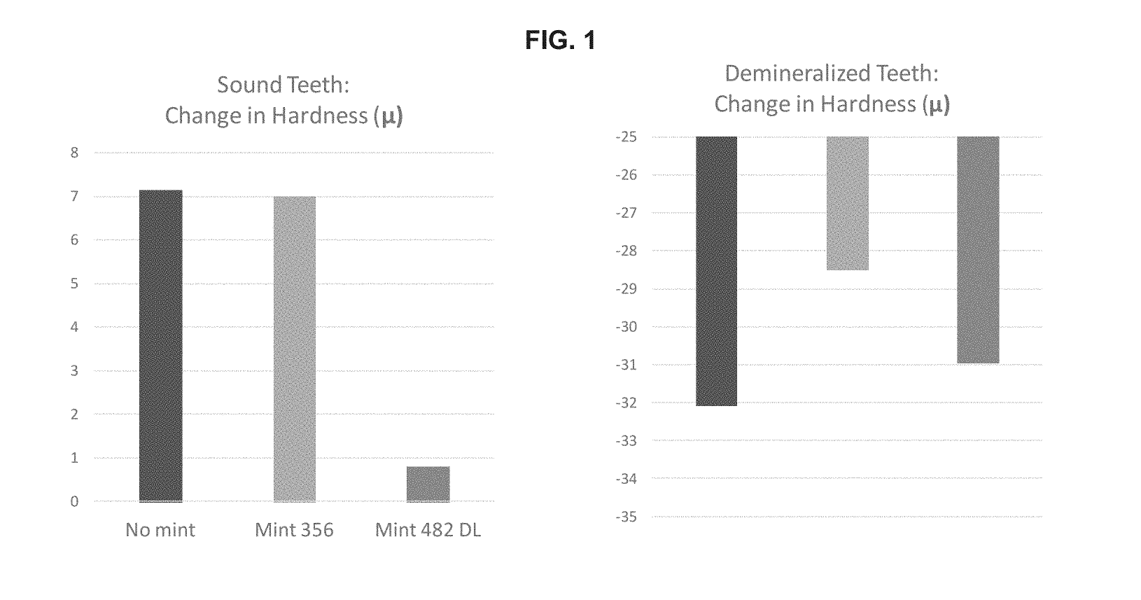

The results of the in situ clinical trial are summarized in FIG. 1. Referring to FIG. 1, the bar charts show the change in tooth hardness measured before and after exposure for each formulation and for the control sample not exposed to either formulation. Sample hardness values were measured by making an indentation in the enamel sample. The larger the indentation, the softer the enamel, as the probe penetrates deeper. The y axis labels relate to the change in the size (in μm) of the indentation. The test caused a slight demineralization, as seen by the larger indentation (+7) in the no mint sample in the chart on the left. Mint 482 remained nearly unchanged, indicating the indentation was a similar size before and after the testing protocol. Negative numbers in the chart on the right show that the indentation became smaller. A larger negative number indicates that the enamel became harder.

The first formulation (designated as “mint 482”) was a dual layer, substantially sugar-free confectionery comprising two adjacent areas of hard candy. The first layer was comprised of approximately 98 percent xylitol as a primary sweetener. The first layer also contained flavorants including peppermint Durarome® encapsulated flavors and other salivation factors. The flavorants were selected to provide a pleasant flavor, physiological cooling effect, and stimulate production of saliva. Durarome is a registered trademark of MCP Foods, Inc. of California, USA.

The mint 482 formulation was prepared according to the process of U.S. Pat. No. 9,179,695 which is incorporated by reference herein. The first layer was partially to mostly crystallized, although it was produced via depositing a melt. The objective was to produce a melt with large enough crystals that they did not remelt and so that it was not necessary to add separate seeding. The first layer was designed to provide an initial burst of cooling and saliva generation in the mouth of a user due, in part, to its negative heat of crystallization. The initial burst of flavor and simultaneous cooling sensation was believed to be key to initiating a rapid spike in salivary flow. It was hypothesized that this process could be enhanced by having the ingredients present in a discrete layer having a relatively rapid dissolution rate.

The second layer was composed of approximately 89 percent Isomalt as a secondary sweetener, a flavorant, and approximately 5 percent hydroxyl propylcellulose (HPC). The second layer was prepared as a polymer melt of secondary sweetener, binder, and flavorants. Isomalt was selected as the secondary sweetener because it melts at a similar temperature to HPC and has a slower dissolution rate than the primary sweetener (xylitol). The goal was to enable the second layer to last in the end-user's mouth after the first layer has dissolved. By so doing, the confectionery would continue to stimulate saliva production after the rapid spike in initial saliva, thereby promoting remineralization in an end-user's mouth for an extended period of time.

HPC was added to the second layer to act as a binder in an attempt to further slow dissolution, add texture, and extend TIM. HPC was selected as the binder because it melts at a similar temperature to Isomalt and it dissolves more slowly.

The second formulation developed for the in situ clinical trial (mint 356) was a pressed tablet mint confection containing a non-homogenous confectionary having a first composition capable of inducing a large and rapid spike in saliva production, combined with a second composition capable of maintaining a steady, long-lasting flow of saliva production. The first composition comprised xylitol at about 13 percent by weight of the total confection while the second composition comprised Isomalt at about 77 percent by weight of the total confection. The composition also contained peppermint Durarome® flavor, salivation factors, citric acid, aspartame, and zinc gluconate.

Again referring to FIG. 1, the results of the in situ trial showed that only the dual layer formulation, mint 482, had a statistically significant effect on preventing demineralization of sound teeth. Neither of these particular formulations showed a statistically significant effect on promoting remineralization of demineralized teeth.

The results of the clinical trial led the present inventors to the unexpected conclusion that increasing salivary flow alone was not sufficient to promote remineralization. Subsequently, the inventors evaluated whether functional active ingredients could be added to the formulation to promote remineralization. Two classes of substances identified for developmental studies were ingredients which could provide a source of calcium, and ingredients to modulate bacterial activity on tooth surfaces. It was believed that bacterial activity at the tooth surface creates a barrier film which inhibits saliva from performing its natural remineralization process. Biofilm is also known to contain acid-producing bacteria. These bacteria, when given a carbohydrate source, as was included in the test protocol, produce acids which promote demineralization. Arginine was known as a dentifrice additive to help disperse biofilm and also to raise pH and promote bonding of calcium to the tooth surface.

In the search for a suitable calcium source, a number of calcium salts were screened for their potential use as an active ingredient in the confectionary formulation. The screening process considered a number of critical factors. A useful candidate would necessarily have approval for use as a food ingredient and to provide acceptable taste. Beyond these threshold criteria, a good candidate would have to be capable of providing sufficient available calcium in salivary solution. In order to be practical and economically viable, the compound would have to be thermally stable over the range of temperatures encountered in the manufacturing process (especially in a melt process) so as to not cause any complications during the manufacturing process for the confections.

The most promising candidates, comprising carbonate, sulfate, gluconate, and glycerophosphate salts of calcium, were included in an in vitro screening study. The in vitro study was designed to evaluate the remineralization efficacy of different calcium salts using the dual layer form of lozenges made by melt processes, since this formulation produced favorable results in preventing demineralization of bovine tooth samples in situ. Each lozenge prepared for the study comprised a fast-dissolving xylitol-based first layer and a slower-dissolving Isomalt and HPC-based second layer. The calcium salt candidates were added to the second layer. The second layer's longer TIM provided the calcium salts with a longer time period to contact the samples.

The in vitro study used bovine tooth enamel slabs which were polished and then subjected to an initial tooth softening step using lactic acid. An initial microhardness measurement was then made on each enamel specimen. The enamel specimens were subjected to four pH cycles per day, each cycle comprising a thirty-minute acid demineralization step followed by a twenty-minute lozenge remineralization step. Final microhardness measurements were taken on each specimen after 10 days of treatment and after 20 days of treatment. A fluoride dentifrice and a no-lozenge control sample were analyzed according to the same procedure for comparison purposes.

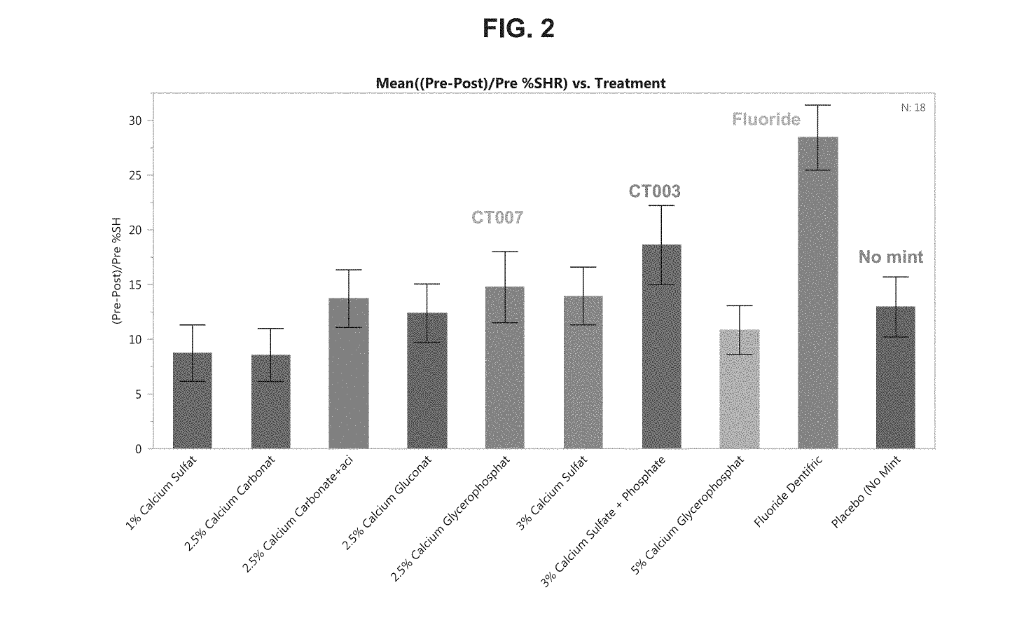

The 20-day results of the in vitro study are shown in FIG. 2. The graph shows the Surface Hardness Recovery (SHR) for samples exposed to the formulations. SHR is the percentage change in hardness (pre-test value minus post-test value) divided by pre-test value.

As expected, referring to FIG. 2, the fluoride treatment produced significantly better results compared to no mint after 10 and 20 days. Two of the calcium salt lozenges in the study revealed potentially significant results. The first contained 2.5 percent calcium glycerophosphate (CGP), the second a 3 percent mixture of calcium sulfate and phosphate, both as percentages of the total lozenge. Although the results for these formulations were noteworthy, they were not appreciably greater than the result obtained with no mint (no confection).

The results of the in vitro study of calcium sources did not yield any formulations with the level of remineralization efficacy the inventors had hoped to discover. This led the present inventors to seek alternative formulations to be used alone or in conjunction with calcium sources to produce the desired goal of a lozenge with remineralization properties. Among the alternative technologies considered was using arginine as an ingredient in the formulation as a way to increase saliva pH, bind calcium to the dentine surface and disperse biofilm. Sodium trimetaphosphate was also considered as a means for replacing dissolved phosphate and to foster biomimetic remineralization of enamel. Chitosan was considered for its ability to inhibit oral bacteria and to complex with and deliver calcium on tooth surfaces. Molecules developed specifically as biofilm dispersing agents were also considered.

The latter two alternatives, chitosan and biofilm dispersing agents, were ruled out because they lacked approval for food use. Arginine was selected as the leading candidate for further evaluation. A new in vitro study was undertaken to evaluate arginine alone and in combination with CGP and HPC. CGP alone, CGP with HPC and fluoride were also investigated. The testing procedure was substantially the same as the in vitro study for calcium salts.

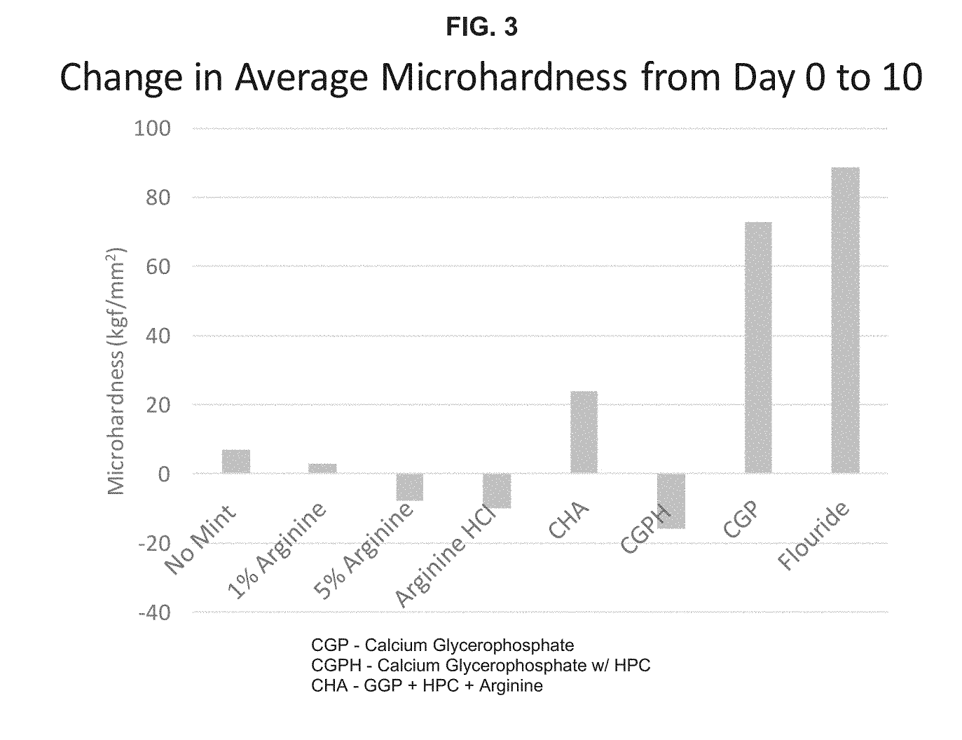

The results of the in vitro study are shown in FIG. 3. The chart in FIG. 3 shows the change in enamel microhardness observed after 10 days of exposure to each formulation, expressed in the units of the Knoop Hardness Number (KHN). The height of each bar represents the change in the KHN as calculated by ΔKHN=KHN_{10-DAY}−KHN_{INITIAL}.

Referring to FIG. 3, the 10-day microhardness results of the second in vitro study unexpectedly showed that CGP produced an increase in microhardness that was very similar to the sample for fluoride. However, CGP combined with HPC yielded a negative hardness result. Therefore, the inventors unexpectedly discovered that HPC, which had been included as a binder and to increase TIM, had a negative impact on remineralization. As a result, HPC was removed from future formulations.

Arginine and arginine HCl did not produce significant results when used alone. However, arginine in combination with CGP and HPC produced an increase in microhardness that was significantly greater than CGP and HPC alone. Based on the results of this study, the present inventors discovered that CGP and CGP in combination with arginine had the potential to provide a mint with the remineralization abilities the inventors were seeking.

In addition to evaluating additives for their ability to provide tooth strengthening properties, the inventors also investigated a number of sugar alcohols, individually and in combination, for their ability to increase salivary flow and TIM. The investigation included dual layer formulations as well as compressed tablets. Greater salivary flow is desirable to create calcium delivery and increased TIM provides greater delivery time for active ingredients to have an effect.

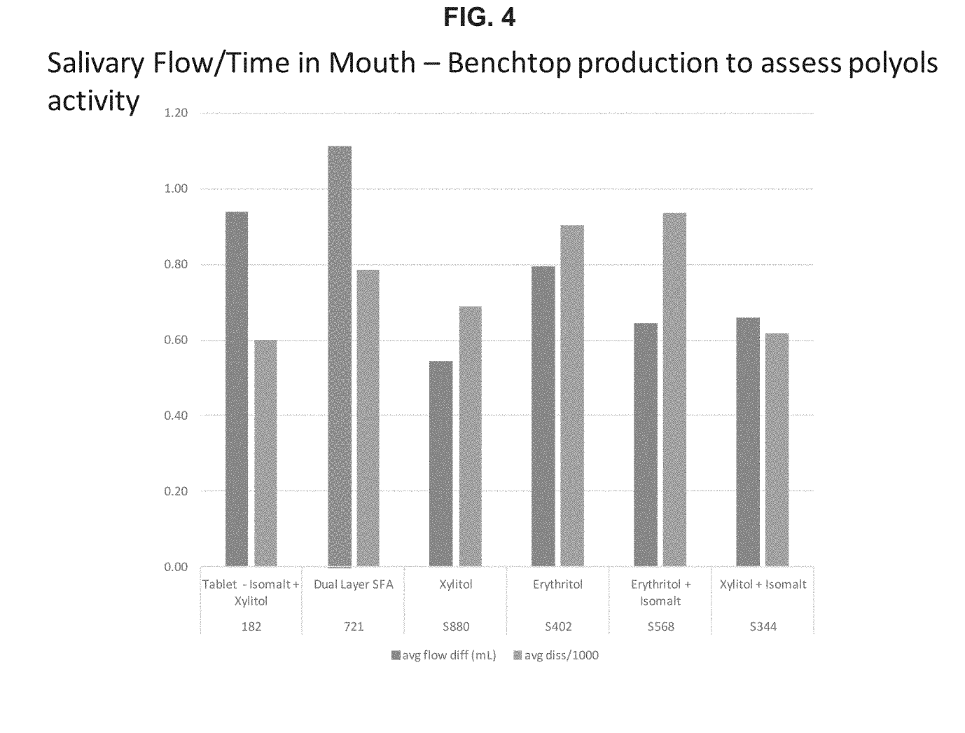

The results of the sugar alcohol assessment are summarized in FIG. 4. In FIG. 4, the blue bars represent the average salivary flow difference (in ml/min) between test subjects at rest with no mint versus test subjects using a mint. The green bars represent the average dissolution time in seconds/1000.

Referring to FIG. 4, the preliminary results of the sugar alcohol assessment indicated that erythritol performed well at both functions. It stimulated salivary flow but had a slower dissolution rate than many of the other sugar alcohols tested.

The results for erythritol provided motivation to substitute erythritol in place of xylitol in existing manufacturing processes for the dual layer and pressed tablet formulations. However, erythritol proved problematic due to its higher temperature of crystallization. Attempts to use erythritol in processes similar to those used for xylitol were challenging. Where it was used in combination with arginine, the higher processing temperatures required by erythritol led to undesirable arginine browning. It became clear to the inventors that if arginine was to be an ingredient in the formulation, a lower temperature process would be required that would be amenable to processes incorporating erythritol.

Arginine presented some other challenges in the manufacturing process. When arginine was mixed into the xylitol layer of the dual layer formulation, it quickly degraded, as evidenced by discoloration. The inventors discovered that arginine HCl could be used in the xylitol layer without any noticeable discoloration. However, the effectiveness of arginine HCl was not as demonstrable in the clinical study results (see FIG. 3). Arginine, and other materials which act via changes in biofilm and its activity, did not show effectiveness in the tests because there was no biofilm present.

A modified method was therefore required for manufacturing a product for delivering arginine. Arginine had been processed successfully in an Isomalt tablet during prior clinical studies. At about the same time, Isomalt was selected as the best tablet formulation excipient based on its dissolution time. Isomalt's relatively slower dissolution rate provides longer TIM which provides more contact time between active ingredients and tooth surfaces. The dissolution rate of the tablet sweetener became a greater consideration after the HPC binder was eliminated from the formulation because of its inhibiting effect on remineralization. As a result of these considerations, arginine was identified as being a well-suited additive for the Isomalt-based tablet formulation.

The inventors next focused on evaluating the effectiveness of compositions for delivering CGP and arginine. In an in situ clinical trial, compositions were tested on bovine enamel using a procedure substantially the same as the prior clinical trials. The trial identified three formulations which were successful at preserving tooth hardness in sound enamel specimens and/or for promoting remineralization of enamel specimens with carious lesions.

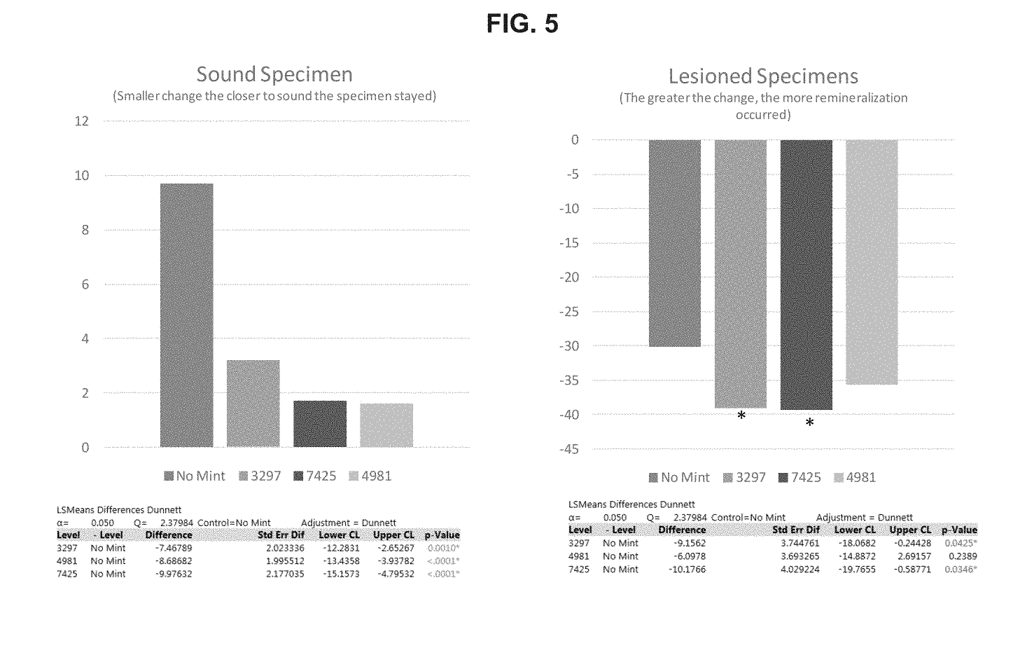

The results of the in situ clinical trial are summarized in FIG. 5. As in FIG. 1, the bar charts in FIG. 5 show the change in tooth hardness measured before and after exposure for each formulation and for the control sample not exposed to either formulation. Sample hardness values were measured by making an indentation in the enamel sample. The larger the indentation, the softer the enamel, as the probe penetrates deeper. The y axis labels relate to the change in the size (in μm) of the indentation. The height of each bar in the chart on the left represents the change in the depth of indentations produced in the hardness tests on sound teeth. Negative numbers in the chart on the right show that the indentation became smaller. A larger negative number indicates that the enamel became harder.

Referring to FIG. 5, one successful formulation, sample 7425, was a dual layer mint comprising a first layer of xylitol and a second layer of Isomalt containing CGP. The Isomalt layer also contained zinc gluconate and medium chain triglycerides as breath freshening agents. The CGP, zinc gluconate and triglycerides were mixed into the Isomalt layer during the processing of the formulation. This formulation produced positive results on both sound enamel samples and samples with carious lesions.

Another formulation with positive results was sample 4981, a dual layer mint comprising all the ingredients of sample 7425 with the addition of arginine HCl in the xylitol layer. Referring to FIG. 5, the trial results showed that this formulation had significant results maintaining the hardness of sound enamel specimens but the results for promoting remineralization of lesioned teeth were statistically insignificant.

A pressed tablet formulation, sample 3297, was successful at preventing demineralization of sound enamel samples and promoting remineralization of lesioned enamel samples. The pressed tablet comprised Isomalt, arginine, CGP, zinc gluconate and triglycerides for breath freshening and flavorings.

As a result of the extensive development effort, the inventors arrived at embodiments of a confectionery product which could effectively deliver oral care benefits, including remineralization, and could be manufactured efficiently with a minimal number of additives. Specifically, the embodiments of the present invention do not require a phosphate salt additive to be effective at promoting remineralization. Embodiments of the present invention are described more thoroughly below.

Dual Area Confection

In an embodiment, the confection having oral care benefits and desirable time in mouth is comprised of two areas. The first area comprises a primary sweetener and the second area comprises a secondary sweetener. Each area may optionally include other ingredients, for example flavorings, colorings and functional active ingredients. The areas may be arranged as layers or sections of a solid confection.

First Area

The first area preferably comprises a primary sweetener, at least one flavorant and, optionally, at least one colorant. The primary sweetener is preferably a sugar alcohol such as: xylitol, maltitol, mannitol, sorbitol, erythritol, arabitol, glycerol, lactitol, and the like. In this embodiment the primary sweetener is xylitol. In an embodiment, the first area is prepared as a polymer melt of primary sweetener, flavorants and optional ingredients. In another embodiment, the first area is prepared as a slurry or any other type of mixture known in the art.

The primary sweetener preferably comprises 60 percent to 100 percent by weight of the first area, more preferably from 85 percent to 100 percent by weight of the first area, most preferably from 95 percent to 100 percent by weight of the first area.

The first area may also contain one or more flavorants. Suitable flavorants include, but are not limited to, food grade acids, essential oils, spice and salt, conventionally used in confectionery products. Particularly preferred flavorants are those derived from mint oils such as peppermint, spearmint and the like because in combination with the sugar alcohol the resulting flavor composite yields a particularly cooling taste sensation.

The flavorants may be in solid form, such as a powder, crystalline, amorphous crystal, semicrystalline and the like. They may be in the form of liquids or they may be encapsulated or they may be spray dried. Additional flavors include those derived from essential oils, as well as those flavors characterized as either natural or artificial flavors. Examples include essential oils such as, without limitation, cinnamon, spearmint, peppermint, birch, and the like; natural or artificial fruit flavors, such as, without limitation, apple, pear, peach, strawberry, cherry, apricot, orange, lemon, watermelon, banana, and the like; bean-derived flavors such as, without limitation, coffee, cocoa powder and the like. The flavoring agent may be a spice commonly used in foods. Examples include chili powder, curry powder and the like. The flavorant may be a salt commonly used in the food arts, such as sodium chloride, potassium iodide, potassium chloride, sodium iodide and the like. In some embodiments, the product of the present invention contains one or more flavorants.

Flavorants may include compounds known to promote salivation. Suitable salivation promoting compounds include, for example, trans-pellitorin extracts of Heliopsis longipes root, extracts of Piper nigrum, alkyloxyalkane acid amides, alk-2-en-4-yne acid amides, food acids, and compounds providing a salty taste.

Flavorants are present in flavoring effective amounts known in the art. Flavorants preferably comprise 0.005 percent to 10 percent by weight of the first area, more preferably from 0.01 percent to 5 percent by weight of the first area, most preferably from 0.01 percent to 2 percent by weight of the first area.

The first area may contain synthetic or natural food grade colorant such as, for example, azo coloring agents or carotenoids (e.g., B carotene, canthaxathin and the like). The colorants preferably comprise up to 5 percent by weight of the first area, more preferably, up to 1 percent by weight of the first area, most up to 0.1 percent by weight of the first area.

The first area of the present invention has very low moisture content. Water is present in an amount of preferably less than 1 percent by weight of the first area, more preferably less than 0.5 percent by weight of the first area, most preferably less than 0.25 percent by weight of the first area.

Second Area

The second area of the dual area embodiment preferably is comprised of a secondary sweetener, at least one flavorant, optionally, one or more functional active ingredients, optionally, one or more colorants, and optionally, one or more breath deodorizers. In an embodiment, the second area is prepared as a polymer melt of secondary sweetener, flavorants and optional ingredients. In another embodiment, the second area is prepared as a slurry or any other type of mixture known in the art.

The secondary sweetener preferably comprises from 80 percent to 100 percent by weight of the second area, more preferably from 85 percent to 100 percent by weight of the second area and most preferably from 90 percent to 100 percent by weight of the second area.

The secondary sweetener preferably has a slower dissolution rate in saliva at normal body temperature than the primary sweetener. This ensures that the second area remains in the end-user's mouth after the first area has dissolved. The confectionery thus continues to stimulate saliva production after the rapid spike in initial saliva production facilitated by the dissolution of the first area, thereby promoting oral care in an end-user's mouth for an extended period of time. If optional functional active ingredients are present, the increased time in mouth lengthens their contact time with the end-user's tooth enamel.

Suitable secondary sweeteners include, but are not limited to, sugar alcohols such as: Isomalt, maltitol syrups, and hydrogenated starch hydrosylate (HSH) syrups. In this embodiment, Isomalt is the secondary sweetener of the second area. The Isomalt component of the second area may be partially to mostly crystallized, or it may be amorphous.

The second area preferably comprises flavorants. Examples of suitable flavorants include, but are not limited to, substantially sugar-free sweeteners such as acesulfame potassium and various aromatic flavors known in the art of confectioneries. Flavorants may be encapsulated in polyol structures such as Durarome® or Flexarome® encapsulated flavors. In this embodiment flavorants include acesulfame potassium and peppermint Durarome® flavor.

Flavorants preferably comprise up to 10 percent by weight of the second area, more preferably up to 7 percent by weight of the second area, most preferably up to 5 percent of the second area.

The second area optionally comprises synthetic or natural food grade colorants such as, azo coloring agents or carotenoids (e.g., B carotene, canthaxathin and the like), or edible pigments known in the art of confectioneries. When present, colorants preferably comprise less than 5 percent by weight of the second area, more preferably less than 1 percent by weight of the second area, most preferably less than 0.5 percent by weight of the second area.

The second area optionally comprises one or more functional active ingredients. Functional active ingredients may include, but are not limited to, calcium ion sources, phosphate ion sources, anti-bacterial compounds and biofilm dispersants. Functional active ingredients provide benefits to tooth enamel by protecting against demineralization, i.e., the breakdown of tooth enamel over time, while promoting remineralization, i.e. repair of enamel loss by replacing lost minerals.

In an embodiment, the functional active ingredient is a calcium-ion releasing compound. Preferred calcium ion-releasing compounds are sparingly soluble calcium-containing salts of biologically-compatible acids and other basic calcium compounds. As used herein, “sparingly soluble” refers to calcium compounds having a solubility greater than about 0.1 g/100 g of water, and less than about 10 g/100 g of water, under conditions of neutral pH and normal human body temperature. Sparingly soluble calcium compounds include, but are not limited to, the calcium salts of gluconate, glycerophosphate, lactate, and fumarate, Ca(OH)₂, CaO monocalcium phosphate, dicalcium phosphate anhydrous, dicalcium phosphate dihydrate, tricalcium phosphate, octacalcium phosphate, tetracalcium phosphate, and combinations and mixtures thereof. In an embodiment, the calcium-ion releasing compound is calcium glycerophosphate.

When present, calcium-ion releasing compounds comprise from 1 percent to 10 percent by weight of the second area, more preferably from 3 percent to 7 percent by weight of the second area, most preferably from 4 percent to 6 percent by weight of the second area.

The second area optionally comprises odor-neutralizing compounds. Odor-neutralizing ingredients known in the confectionery arts are suitable for use in the present invention, including, for example, metal ion-releasing compounds where the metal is copper, magnesium, sodium, tin or zinc. In an embodiment, the odor neutralizing compound is zinc gluconate.

When present, odor-neutralizing compounds comprise from 0.1 percent to 1 percent by weight of the second area, more preferably from 0.2 percent to 0.8 percent by weight of the second area, and most preferably from 0.4 percent to 0.6 percent by weight of the second area.

The second area may optionally include one or more food additives normally found in confections such as preservatives, food grade processing agents and other food additives typically used in confectionery products. Examples include, but are not limited to, the food grade processing agent magnesium stearate and polishing agents containing medium chain triglycerides, such as Neobee®. Neobee is a registered trademark of Stepan Company of Illinois, USA.

When present, food additives comprise up to 4 percent by weight of the second area, more preferably up to 2 percent by weight of the second area, most preferably up to 1 percent by weight of the second area.

Processes of manufacturing some embodiments of the dual area confectionery are disclosed in U.S. Pat. No. 9,179,695, which is incorporated herein by reference. The dual area confectionery can be made via an extrusion and depositing process where a mass is first mixed and melted in an extruder, then charged to a depositor with moulds. The confectionery is produced in two steps. The first step involves formulating the second area and depositing it into a mould, followed by formulating and depositing the first area, per the process described in U.S. Pat. No. 9,179,695, on top of the second area and allowing it to set. This depositing order can be reversed, however, so that the first area is deposited first, and the second area is deposited on top thereof. A dual depositor system is another suitable method of producing the dual area confectionery, thereby allowing for any orientation and order of the two areas, such as the first area being provided as a coating surrounding all or part of the second area, for example.

In an embodiment, the first area is produced by subjecting the composition comprising the primary sweetener to extrusion inside an extrusion apparatus under conditions to partially melt the primary sweetener to form a slurry and maintain this composition throughout the extrusion process. The slurry is formed into a product of desired shape and cooled to form a non-compressible comestible product. The primary sweetener is not fully melted at any time during the process.

As used herein, “fully melted” means that 100 percent of the primary sweetener changes from a crystalline solid into a molten state. In an embodiment of the present process, at least 10 percent by weight of the solid primary sweetener is melted, while in another embodiment at least 50 percent by weight of the solid is melted. In a still further embodiment, at least 90 percent of the solid is melted. In a further embodiment, at least 95 percent of the solid is melted. As indicated hereinabove, in each of the embodiments in the present process, in the extruder, not all of the solid is melted, that is, less than 100 percent of the solid primary sweetener is melted.

As used herein, “slurry” means a translucent mass in a slush-like state. As a slurry, the composition which forms the first area contains a heterogeneous mixture of the ingredients described hereinabove in solid and liquid form. For example, it comprises primary sweetener solid mixed with primary sweetener liquid and any optional ingredients. As indicated above, the mixture remains a slurry throughout the extrusion process.

In an embodiment, the first area is prepared as a slurry in the same manner, using primary sweetener in place of the secondary sweetener. In an embodiment, the first area and/or second area is extruded under conditions to form a polymer melt.

Compressed Confection

In another embodiment of the present invention, a long-lasting confectionery product with oral care benefits is prepared in the form of a compressed confection (also referred to herein as a “pressed tablet” made by known tablet press methods and equipment, such as in pressed mint confections). The compressed confection is comprised of a sugar-free sweetener, and optionally, at least one flavorant, and, also optionally, one or more functional active ingredient(s).

Suitable sugar-free sweeteners include, but are not limited to, sugar alcohols such as: Isomalt, Maltitol syrups, hydrogenated starch hydrosylate (HSH) syrups, and the like. A particularly preferred sweetener for use in the compressed confection is Isomalt.

Preferably the sweetener comprises from 75 percent to 100 percent by weight of the confectionery. More preferably, the sweetener comprises from 80 percent to 100 percent by weight of sweetener, most preferably from 85 percent to 95 percent by weight of the compressed confection.

When one or more flavorants are present, they preferably comprise (cumulatively) up to 10 percent by weight of the compressed confectionery. More preferably, flavorants comprise up to 7.5 percent by weight of the confectionery, and most preferably up to 5 percent by weight of the compressed confection. Suitable flavorants include, but are not limited to, substantially sugar-free sweeteners such as aspartame potassium and various aromatic flavors known in the art of confectioneries. Flavorants may be encapsulated in polyol structures such as Durarome® or Flexarome® flavors. Flexarome is a registered trademark of Firmenich SA of Switzerland. Any of the flavorants described as suitable for the dual area embodiments are also suitable for the compressed tablet embodiments. The flavorants may comprise salivation promoting compounds. Suitable salivation promoting compounds include, for example, trans-pellitorin extracts of Heliopsis longipes root, extracts of Piper nigrum, alkyloxyalkane acid amides, alk-2-en-4-yne acid amides, food acids, and compounds providing a salty taste.

Functional active ingredients may be present including, for example, calcium-ion sources, anti-bacterial compounds, biofilm dispersing agents, and calcium-dentin bond promoters. These functional active ingredients provide multiple benefits to an end-user's oral health. These additives are effective in protecting against demineralization, i.e., the breakdown of tooth enamel over time, while promoting remineralization, i.e. repair of enamel loss by replacing lost minerals.

When present, the functional active ingredients preferably comprise (cumulatively) up to 15 percent by weight of the compressed confectionery. More preferably, the functional active ingredients comprise up to 10 percent by weight of the compressed confectionery, most preferably up to 5 percent of the compressed confectionery.

In this embodiment the functional active ingredient is a calcium-ion releasing compound. Preferred calcium ion-releasing compounds are sparingly soluble calcium-containing salts of biologically-compatible acids and other basic calcium compounds. As used herein, “sparingly soluble” refers to calcium compounds having a solubility greater than 0.1 percent and less than 10 percent under conditions of neutral pH and normal human body temperature, where the percentages are expressed as a mass ratio of solute to solvent. Sparingly soluble calcium compounds include, but are not limited to, the calcium salts of gluconate, glycerophosphate, lactate, and fumarate, Ca(OH)₂, CaO monocalcium phosphate, dicalcium phosphate anhydrous, dicalcium phosphate dihydrate, tricalcium phosphate, octacalcium phosphate, tetracalcium phosphate, and combinations and mixtures thereof. In this embodiment, the calcium-ion releasing compound is calcium glycerophosphate.

In this embodiment, the calcium-ion releasing compounds comprises from 0.2 percent to 10 percent by weight of the compressed confectionery. More preferably, calcium-ion releasing compounds comprise from 0.5 percent to 6 percent by weight, most preferably from 1 percent to 4 percent by weight of the compressed confectionery.

Embodiments of the compressed confection may comprise one or more compounds which disperse biofilm. Suitable biofilm dispersing compounds include, but are not limited to, molecules purposefully designed to break up biofilm or naturally occurring amino acids such as arginine. In this embodiment the biofilm dispersing compound is arginine. Preferably, the biofilm dispersing compound comprises from 0.1 percent to 5 percent by weight of the compressed confectionery. More preferably, the biofilm dispersing compound comprises from 0.25 percent by weight to 3 percent by weight of the confection. Most preferably the biofilm dispersing compound comprises from 0.5 percent by weight to 2 percent by weight of the confectionery.

The compressed confectionery optionally comprises odor-neutralizing compounds. Odor-neutralizing ingredients known in the confectionery arts are suitable for use in the present invention, including for example, metal ion-releasing compounds where the metal is copper, magnesium, sodium, tin or zinc. In this embodiment the odor neutralizing compound is zinc gluconate. When present, odor-neutralizing compounds comprise from 0.01 percent to 1 percent by weight of the compressed confectionery, more preferably from 0.03 percent to 0.7 percent by weight of the compressed confectionery, and most preferably from 0.05 percent to 0.5 percent by weight of the compressed confectionery.

The compressed confectionery may comprise one or more colorants. Suitable colorants for use in the compressed confection include those edible pigments known in the art of confectioneries. Any of the colorants described in reference to the dual area embodiment are suitable for use in the compressed tablet embodiment.

The compressed confection may optionally include food additives normally found in confections such as preservatives, food grade processing agents and polishing agents typically used in confectionery products. Examples include, but are not limited to, the food grade processing agent magnesium stearate and polishing agents containing medium chain triglycerides. In this embodiment, the compressed confectionery comprises the food additives magnesium stearate and medium chain triglycerides. The food additives comprise (cumulatively) up to 4 percent by weight of the compressed confectionery. More preferably, the food additives comprise up to 2 percent by weight of the compressed confectionery. Most preferably, the food additives comprise up to 1 percent by weight of the compressed confectionery.

The compressed confection is provided as a tablet. The tablet is formed by combining the ingredients, in powder form, using methods and devices well known in the art for manufacturing confectioneries in compressed tablet form.

### Example 1

Example 1 is a dual area confectionary embodiment of the present invention. The dual area confectionery was made via an extrusion and depositing process where a mass was first mixed and melted in an extruder, then charged to a depositor with moulds. The confectionery was produced in two steps. The first step involved formulating the second area and depositing it into a mould, followed by formulating and depositing the first area, per the process described in U.S. Pat. No. 9,179,695, on top of the second area and allowing it to set.

To form the second area, standard crystalline Isomalt (90.315 percent w/w) with an average particle size distribution of between 0.2 mm and 0.7 mm (Isomalt ST-F) was dry blended at ambient temperature with peppermint Duraromes® (4.000 percent w/w), calcium glycerophosphate (5.000 percent w/w), zinc gluconate (0.200 percent w/w), Neobee® medium chain triglycerides (0.220 percent w/w), acesulfame potassium (0.250 percent w/w) and the flavorings Symrise S.A. (0.010 percent w/w) and Takasago S.A. 0.005 percent w/w), where all percentages are of the second area. The dry ingredients were blended until a homogenous mixture was obtained. The homogenous dry blend was then melted in an extruder then charged to a depositor with moulds where it was allowed to cool and set. Symrise is a registered trademark of Symrise AG, of Germany. Takasago is a registered trademark of Takasago International Corporation of Japan.

To form the first area, granular xylitol (98.985 percent w/w) was dry blended with blue Duraromes® (1.000 percent w/w), Symrise S.A. (0.010 percent w/w) and Takasago S.A. (0.005 percent w/w), where all percentages are of the first area. The mixed ingredients were dry blended until a homogenous mixture was formed. The homogenous mixture was then fed to an extruder having multiple temperature-controlled zones where the xylitol was partially melted according to the process referenced above, to form a slurry. The slurry was then deposited into moulds pre-filled with the already set second area.

### Example 2

Example 2 is a pressed tablet embodiment of the present invention. The pressed tablet was formed by mixing dry powder ingredients, filling a die mold of a tablet press with the mixture, then compressing and ejecting the composition in tablet form.

To create the dry powder, Isomalt (91.475 w/w) suitable for direct compression (Isomalt DC101) was combined with peppermint Duraromes® (4.000 percent w/w), arginine (1.000 percent), calcium glycerophosphate (2.500 percent), zinc gluconate (0.100 percent), Neobee® medium chain triglycerides (0.110 percent w/w), Symrise S.A. (0.010 percent w/w), Takasago S.A. (0.005 percent w/w), magnesium stearate (0.500 percent w/w) and aspartame (0.300 percent w/w). The mixed ingredients were dry blended until a homogenous mixture was formed. The homogenous mixture was then fed to the die mold of a tablet press where it was compressed and ejected in tablet form.

### Example 3

Example 3 is a dual area embodiment of the present invention.

To form the second area, standard crystalline Isomalt (95.315 percent w/w) with an average particle size distribution of between 0.2 mm and 0.7 mm (Isomalt ST-F) was dry blended at ambient temperature with peppermint Duraromes® (4.000 percent w/w), zinc gluconate (0.200 percent w/w), Neobee® medium chain triglycerides (0.220 percent w/w), acesulfame potassium (0.250 percent w/w) and the flavorings Symrise S.A. (0.010 percent w/w) and Takasago S.A. 0.005 percent w/w), where all weight percentages are percentages of the second area. The dry ingredients were blended until a homogenous mixture was obtained. The homogenous dry blend was then melted in an extruder then charged to a depositor with moulds where it was allowed to cool and set.

To form the first area, granular erythritol (82.985 percent w/w) was dry blended with Litesse® Ultra, a hydrogenated polydextrose (10.000 percent w/w), calcium glycerophosphate (5.000 percent w/w), white Duraromes® (2.000 percent w/w), Symrise S.A. (0.010 percent w/w) and Takasago S.A. (0.005 percent w/w), where all weight percentages are percentages of the first area. Litesse is a registered trademark of Dupont Nutrition Biosciences of Denmark. The mixed ingredients were dry blended until a homogenous mixture was formed. The homogenous mixture was then fed to an extruder having multiple temperature-controlled zones where the xylitol was partially melted according to the process referenced above, to form a slurry. The slurry was then deposited into moulds pre-filled with the already set second area.

Although exemplary embodiments of the herein described composition and method have been described in detail above, those skilled in the art will readily appreciate that many additional modifications are possible in the exemplary embodiment without materially departing from the novel teachings and advantages of the herein described composition and method. Accordingly, these and all such modifications are intended to be included within the scope of the herein described composition and method.

1. A confectionery comprising:
a first area comprising a first sugar alcohol; and
a second area distinct from the first area, the second area comprising a second sugar alcohol and at least one flavorant;
wherein each of the first and second sugar alcohols has a dissolution rate in saliva at normal human body temperature, and
wherein the first sugar alcohol dissolution rate is greater than the second sugar alcohol dissolution rate,
and wherein the confectionery provides oral care benefits to an end user, the oral care benefits including at least one of increased salivary flow, dispersion of oral biofilms, remineralization of tooth enamel, and reduced demineralization of tooth enamel.

2. The confectionery of claim 1 wherein the first sugar alcohol is selected from the group consisting of xylitol and erythritol, and comprises 60 percent to 100 percent by weight of the first area.

3. The confectionery of claim 1 wherein the first area is partially crystallized, and wherein the first area has a moisture content of less than 1 percent by weight, based on the total weight of the first area.

4. The confectionery of claim 1 wherein the second sugar alcohol is Isomalt; wherein the Isomalt comprises 80 to 95 percent by weight of the second area.

5. The confectionery of claim 1 wherein the flavorant comprises up to 10 percent by weight of the second area.

6. The confectionary of claim 1 wherein the second area further comprises at least one functional active ingredient chosen from the group consisting of calcium ion-releasing compounds, phosphate ion-releasing compounds, biofilm dispersants, and combinations thereof; the at least one functional active ingredient comprising, in aggregate, 3 to 9 percent by weight of the second area.

7. The confectionery of claim 6 wherein the functional active ingredient is arginine, and wherein the second area of the confection does not comprise hydroxypropylcellulose.

8.-10. (canceled)

11. A confectionery comprising:
a blended mixture comprising:
(a) a first composition having a first sugar alcohol; and
(b) a second composition having a second sugar alcohol, an optional flavorant, and an optional functional active ingredient;
wherein each of the first and second sugar alcohol has a dissolution rate in saliva at normal human body temperature, wherein the first sugar alcohol dissolution rate is greater than the second sugar alcohol dissolution rate,
and wherein the confectionery provides oral care benefits to an end user, the oral care benefits including at least one of increased salivary flow, dispersion of oral biofilms, remineralization of tooth enamel, and reduced demineralization of tooth enamel.

12. The confectionery of claim 11 wherein the functional active ingredient comprises at least one of calcium glycerophosphate and arginine.

13. The confectionery of claim 11, wherein the confectionery is in the form of a pressed tablet.

14. The confectionery of claim 11, wherein the first sugar alcohol is erythritol.

15. A confectionery comprising:
a first sweetener;
a salt comprising calcium ions and phosphate ions, the salt being at least sparingly soluble; and
at least one selected from the group of a second sweetener and biofilm dispersing agent;
wherein the first sweetener has a slower rate of dissolution in human saliva than the second sweetener.

16. The confectionery of claim 15, wherein the salt comprises at least one selected from the group of calcium glycerophosphate, monocalcium phosphate, dicalcium phosphate anhydrous, dicalcium phosphate dihydrate, tricalcium phosphate, octacalcium phosphate, tetracalcium phosphate, and combinations thereof.

17. The confectionery of claim 15, wherein the salt comprises calcium glycerophosphate.

18. The confectionery of claim 15, wherein the salt comprises from 1 percent to 4 percent by weight of the confectionery.

19. The confectionery of claim 15, wherein the biofilm dispersing agent, when present, comprises arginine.

20. The confectionery of claim 15, wherein the biofilm dispersing agent, when present, comprises between 0.5 percent and 2 percent by weight of the confection.

21. The confectionery of claim 15, wherein the first sweetener comprises isomalt.

22. The confectionery of 15, wherein the first sweetener comprises from 75 percent to 100 percent by weight of the confectionery.

23. The confectionery of claim 15, wherein the second sweetener, when present, comprises at least one selected from the group of xylitol and erythritol.
